(19) United States
(12) Patent Application Publication
(10) Pub. No.: US 2020/0283414 A1
(43) Pub. Date: Sep. 10, 2020

(54) Process for preparing crystalline Tipiracil Hydrochloride

(71) Applicants: Nandi SUKUMAR, Hyderabad (IN); Dhanraj THERIVIAM SUDALAY SUNDARAM, Hyderabad (IN); Sreedhar Reddy DWARKAPALLY, Hyderabad (IN); Ravi Kumar Gupta MIRIYALA, Hyderabad (IN); Sivakumaran MEENAKSHISUNDRAM, Hyderabad (IN); AUROBINDO PHARMA LIMITED, Hyderabad (IN)

(72) Inventors: Nandi Sukumar, Hyderabad (IN); Dhanraj Theriviam Sudalaya Sundaram, Hyderabad (IN); Sreedhar Reddy Dwarkapally, Hyderabad (IN); Ravi Kumar Gupta Miriyala, Hyderabad (IN); Sivakumaran Meenakshisunderam, Hyderabad (IN)

(73) Assignee: Aurobindo Pharma Ltd, Hyderabad (IN)

(21) Appl. No.: 16/646,081

(22) PCT Filed: Sep. 10, 2018
(86) PCT No.: PCT/IB2018/056873

§ 371 Date: Mar. 10, 2020

(30) Foreign Application Priority Data
Sep. 14, 2017 (IN) 201741032555

(51) Int. Cl. C07D 403/06

(52) U.S. Cl. CPC C07D 403/06; C07B 2200/13

(57) ABSTRACT

The present invention relates to a process for the preparation of Tipiracil hydrochloride crystal III, which comprises reaction of Tipiracil with hydrochloric acid in presence of solvent is selected from alcohol and/or water. Further, the present invention relates to pure Tipiracil base having purity greater than about 99.0% by HPLC.

### FIELD OF THE INVENTION

The present invention relates to a process for the preparation of Tipiracil hydrochloride crystal III.

Further, the present invention relates to pure Tipiracil base having purity greater than about 99.0% by HPLC.

### BACKGROUND OF THE INVENTION

Tipiracil hydrochloride is chemically known as 5-chloro-6-[(2-imino-1-pyrrolidinyl) methyl]-2,4(1H, 3H)-pyrimidinedione hydrochloride (1:1) and has the following structural formula I:

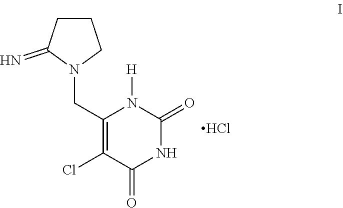 [SMILES: N=C1CCCN1Cc1[nH]c(=O)[nH]c(=O)c1Cl ; Cl]

Tipiracil hydrochloride is marketed in combination with Trifluridine as a metastatic colorectal cancer drug with the trade name of Lonsurf®. Lonsurf® is used for the treatment of patients with metastatic colorectal cancer who have been previously treated with fluoropyrimidine-, oxaliplatin- and irinotecan-based chemotherapy, an anti-VEGF biological therapy, and if RAS wild-type, an anti-EGFR therapy.

WO 96/30346 describes Tipiracil hydrochloride and its preparation. The process involves reaction of 5-chloro-6-chloromethyluracil with 2-iminopyrrolidine in presence of sodium ethoxide and N,N-dimethylformamide to provide Tipiracil, which is then reacted with 1N hydrochloric acid and treated the solution with activated carbon. The obtained mixture is filtered and concentrated the filtrate under reduced pressure to get residue, which is then washed with ethanol to provide Tipiracil Hydrochloride.

Bioorganic & Medicinal Chemistry (2004) 12, 3443-3450 discloses a process for preparing Tipiracil hydrochloride, which involves reaction of 2-iminopyrrolidine hydrochloride with 5-chloro-6-chloromethyluracil in presence of 1,8-diazabicyclo[5.4.0]undec-7-ene (DBU) and methanol to provide Tipiracil, which is dissolved in 2 N hydrochloric acid at a temperature of 90° C. Ethanol is added to the reaction liquid and allowed the reaction mixture to stand at room temperature to get white crystals of Tipiracil hydrochloride.

US 2016/0145241 (the US '241) describes crystal I, crystal II and crystal III, and process thereof. The process for crystals III involves dissolution of Tipiracil in hydrochloric acid and water to provide solution, which is filtered and concentrated. Ethanol is added to the concentrated reaction mixture at room temperature to get Tipiracil hydrochloride crystals III. In another process of US '241 for Crystal III involves dissolution of Tipiracil in ethanol and concentrated hydrochloric acid. The mixture is stirred at a temperature of 64° C. for 1 hour and is cooled to a temperature of 30° C. The resulting crystals were separated by filtration and washed with methanol to get Tipiracil hydrochloride crystals III.

Further, the US '241 states the crystal III of Tipiracil hydrochloride contains the amount of ethanol is more than 16000 ppm and methanol is more than 49862 ppm. This value exceeds the reference value (5000 ppm) of ethanol and (3000 ppm) of methanol described in the Guideline for Residual Solvents in ICH guidelines.

Generally, when a compound is used as an active ingredient for medicaments, the compound is required to have chemical and physical stability for preservation of stable quality and/or easy storage and management. For the reason, such a compound is preferably produced in a stable crystal form. Also, when a compound is used as an active pharmaceutical ingredient in a drug, the most stable crystal form of the compound is selected. Moreover, Guideline for Residual Solvents in ICH (International Conference on Harmonisation) makes recommendations regarding which of various solvents should be avoided/limited/used in the acceptable amounts thereof. Some solvents used in producing medicaments are toxic, and therefore, in view of safety, the amount of such a solvent remaining after a production process is desirably as small as possible.

Organic solvents are frequently used during processing of chemical materials, and subsequent removal of solvents is one of key steps for the production of pure chemical products. Drying is typically used, but in some cases it is difficult to remove residual solvents by drying especially if solid material or when particles are obtained that is big, irregular, agglomerated and may be because of the limited stability. These solvents must be reduced to levels that are acceptable for pharmaceutical use. The pharmaceutically acceptable level depends upon the solvent and the maximum daily dose to be administered. Guidelines for what is acceptable are provided by ICH.

Based on present inventors observation while developing Tipiracil Hydrochloride that the compound obtained from the above processes of the prior art contain more residual solvent and the removal of these solvents from Tipiracil Hydrochloride either by milling, drying at higher temperature or by co-distillation with solvents is difficult without affecting the purity of Tipiracil Hydrochloride.

Therefore, there is a need to develop an alternative process for the preparation of crystal III of Tipiracil hydrochloride, which is simple and controls/reduces the residual content of solvent used in the final API.

### OBJECTIVE OF THE INVENTION

The objective of the present invention is to provide a process for preparing crystal III of Tipiracil hydrochloride.

Another objective of the present invention is to provide Tipiracil base having purity greater than about 99.0% by HPLC.

### SUMMARY OF THE INVENTION

In an aspect of the present invention, there is provided Tipiracil Hydrochloride Crystal III, which is substantially free of residual solvent.

In another aspect of the present invention, there is provided a process for the preparation of crystal III of Tipiracil hydrochloride substantially free from residual solvent comprises reaction of Tipiracil with hydrochloric acid in presence of solvent system comprises alcohol and water.

In another aspect of the present invention, there is provided a process for the preparation of crystal III of Tipiracil hydrochloride substantially free from residual solvent comprises reaction of Tipiracil with hydrochloric acid in presence of C₄-alcohol.

In another aspect of the present invention, there is provided Tipiracil base having purity greater than about 99.0% by HPLC.

In another aspect of the present invention, there is provided Tipiracil base having purity greater than about 99.0% by HPLC, which comprises treatment of crude Tipiracil base with an acid followed by a base.

### BRIEF DESCRIPTION OF THE DRAWINGS

FIG. 1 is powder X-ray power diffraction (“PXRD”) pattern of crystal III of Tipiracil hydrochloride prepared according to the Example 4.

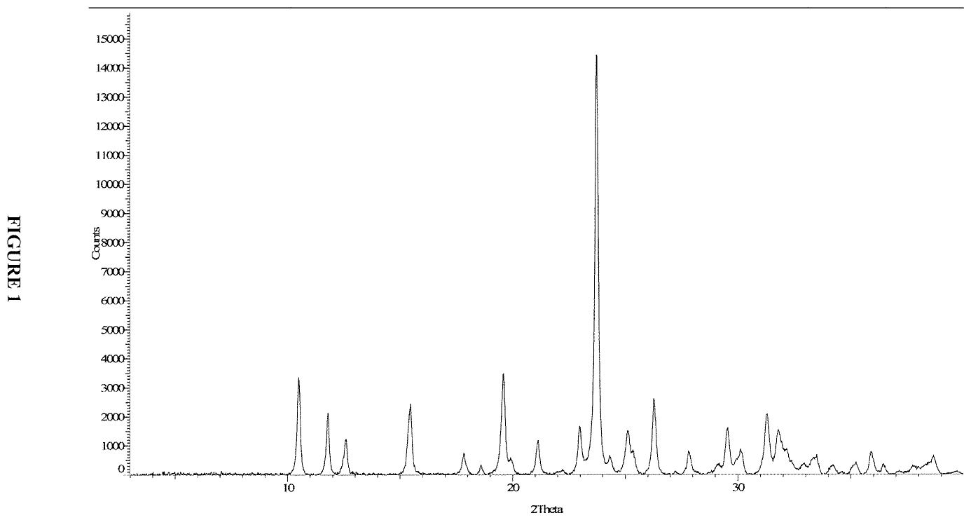

FIG. 2 is powder X-ray power diffraction (“PXRD”) pattern of Tipiracil hydrochloride prepared according to the Example 5.

FIG. 3 is powder X-ray power diffraction (“PXRD”) pattern of Tipiracil base prepared according to the Example 2.

### DETAILED DESCRIPTION OF THE INVENTION

In an aspect of the present invention, there is provided Tipiracil Hydrochloride Crystal III, which is substantially free of residual solvent.

The phrase “Substantially free of residual organic solvent” as referred herein means the solvent content in the API is as per the limit of ICH guidelines.

In another aspect of the present invention, there is provided a process for the preparation of crystal III of Tipiracil hydrochloride substantially free from residual solvent comprises reaction of Tipiracil with hydrochloric acid in presence of solvent system comprises alcohol and water.

The suitable alcohol comprises methanol, ethanol, isopropanol, propanol, 1-butanol, tertiary butanol or mixtures thereof. In an embodiment, the alcohol comprises 1-butanol, tertiary butanol or mixture thereof.

The reaction of Tipiracil and hydrochloric acid is performed by the addition of hydrochloric acid to the reaction mixture at a temperature range of about 0 to 100° C. or reflux temperature of the solvents used or any other suitable temperature. In an embodiment, the temperature is selected from 20 to 50° C. for the reaction.

The process for the preparation of crystal III of Tipiracil hydrochloride involves dissolution of Tipiracil base in a mixture of water and alcohol followed by the addition of hydrochloric acid to provide Crystal III.

The solution may be filtered to remove any insoluble particles. The solution may be filtered by passing through paper, glass fiber, or other membrane material, or a bed of a clarifying agent such as Celite® or Hyflow. Depending upon the equipment used and the concentration and temperature of the solution, the filtration apparatus may need to be preheated to avoid premature crystallization.

The addition of hydrochloric acid to the reaction mixture is performed at a temperature of less than about 60° C. or less than about 50° C. or less than about 40° C. or less than about 30° C. or less than about 20° C. or reflux temperature of the solvents used or any other suitable temperature to obtain suspension of Tipiracil Hydrochloride. The hydrochloric acid used can be aqueous HCl or alcoholic HCl.

Optionally, the solid can be isolated by the removal of solvents from the solution, suspension or dispersion obtained from step b) by techniques known in the art such as distillation, evaporation, oven drying, tray drying, rotational drying (such as the Buchi Rotavapor), spray drying, freeze-drying, fluid bed drying, flash drying, spin flash drying and Ultrafilm agitated thin film dryer-vertical (ATFD-V), hot melt extrusion (HME) and the like.

The crystals thus obtained may be washed with an organic solvent includes C₁-C₆ alcohol or lower alcohol, for example, methanol, ethanol, and the like; and combinations thereof.

The solid obtained may be dried. The drying temperature chosen can be at least 20° C., preferably at least 35° C., but can also be higher like for example at least 40° C., 50° C. or at least 60° C.

In another aspect of the present invention, there is provided a process for the preparation of crystal III of Tipiracil hydrochloride substantially free from residual solvent comprises reaction of Tipiracil with hydrochloric acid in presence of C₄-alcohol.

The C₄-alcohol comprises n-butanol, isobutanol or tertiary butanol. The reaction between Tipiracil base and hydrochloric acid is performed at a temperature of about 0 to 100° C. or at reflux temperature based on the solvent used.

In another aspect of the present invention, there is provided Tipiracil base having purity greater than about 99.0% by HPLC, which comprises treatment of crude Tipiracil base with an acid followed by a base.

The suitable acid comprises organic acid or inorganic acid. The organic acid is selected from formic acid, oxalic acid, acetic acid, 2,2-dichloroacetic acid, adipic acid, ascorbic acid, aspartic acid, benzenesulfonic acid, benzoic acid, 4-acetamido-benzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, salicyclic acid, 4-aminosalicyclic acid, sebacic acid, stearic acid, succinic acid, tartaric acid, thiocyanic acid, toluenesulfonic acid, and undecylenic acid. The inorganic acid is selected from hydrochloric acid, sulfuric acid, phosphoric acid or mixtures thereof. In an embodiment of the present invention, the acid is acetic acid.

The suitable base comprises inorganic base or organic base. The inorganic base is selected from alkaline metal hydroxides such as lithium hydroxide, sodium hydroxide and potassium hydroxide; alkaline metal bicarbonates such as sodium bicarbonate and potassium bicarbonate. The organic base is selected from secondary and tertiary organic amines such as triethylamine (TEA), N,N-diethylisopropylamine, N,N-diisopropylethylamine (DIPEA), diethylamine, tripropylamine and trioctylamine. In an embodiment of the present invention, the base is sodium hydroxide.

The purification process of Tipiracil base comprises suspending Tipiracil crude in water, treating the reaction mass with an acid to obtain a clear solution followed by adjusting the pH of reaction solution with base.

The purification of Tipiracil crude is performed at a temperature range of about 0 to 100° C. or more based on the acid and base used.

The tipiracil base thus obtained may be dried at a temperature of about 20° C. to about 60° C. or more without affecting the purity of the desired product and then converted to Tipiracil hydrochloride.

The tipiracil base thus obtained from the present invention has purity greater than about 95.0% or greater than 99.0% by HPLC.

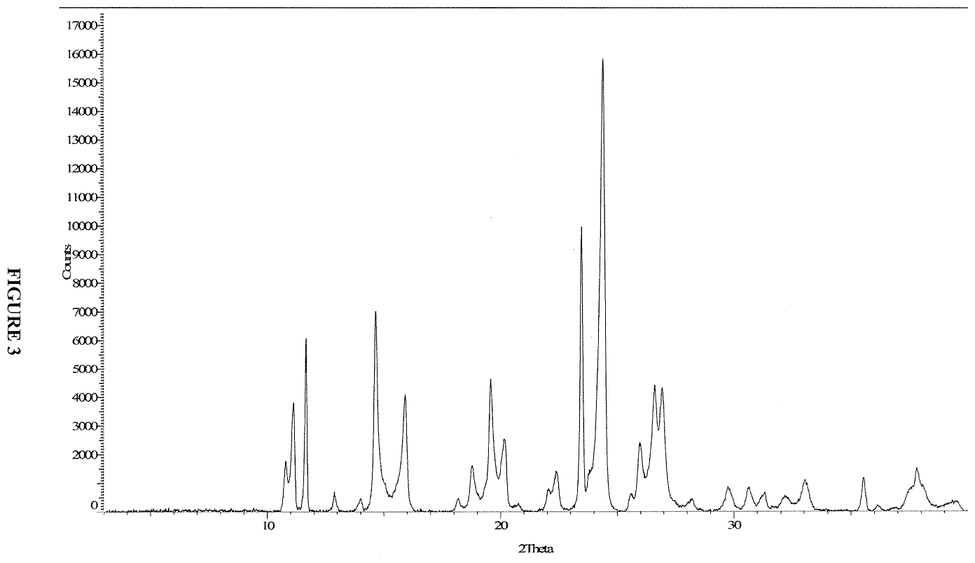

In an embodiment of the present invention, there is provided crystalline Tipiracil base, which is characterized by X-ray powder diffraction pattern represented as FIG. 3.

Having described the invention with reference to certain aspects embodiments, embodiments will become apparent to one skilled in the art from consideration of the specification. The invention is further defined by reference to the following examples describing the preparation of crystalline Tipiracil hydrochloride Crystal III and purification of Tipiracil base having purity greater than about 99.0% by HPLC. It will be apparent to those skilled in the art that many modifications, both to materials and methods, may be practiced without departing from the scope of the invention.

### EXAMPLE 1

### PREPARATION OF 5-CHLORO-6-[(2-IMINO-1-PYRROLIDINYL) METHYL]-2,4(1H, 3H)-PYRIMIDINEDIONE (TIPIRACIL CRUDE)

To a stirred solution of 2-iminopyrrolidine hydrochloride (79.9 g; 0.66 mol) in methanol (1000 ml), DBU (194.9 g; 1.28 mol) was added at 25-30° C. Thereafter, 5-Chloro-6-chloromethyluracil (100 g; 0.51 mol) was added and stirred the suspension at 60-65° C. for 4-5 h. The reaction mixture was cooled to 25-30° C. and stirred for another 2-3 h. The solid product was filtered, washed with methanol (300 ml) and dried under reduced pressure (10-20 mmHg) at 45-50° C. to obtain Tipiracil crude.

Yield: 98.2 g

Chromatographic purity by HPLC: >98%

### EXAMPLE 2

### PURIFICATION OF 5-CHLORO-6-[(2-IMINO-1-PYRROLIDINYL) METHYL]-2,4(1H, 3H)-PYRIMIDINEDIONE (TIPIRACIL)

Tipiracil crude (90 g; 0.37 mol) was suspended in DM water (900 ml) at 25-30° C. The pH of above suspension was adjusted to 4.0-4.2 using glacial acetic acid (50 ml) at 25-30° C. to obtain clear solution. Further the solution was neutralized with 10% sodium hydroxide solution (pH: 6.5-7.0) at 25-30° C., during which product precipitates out. Thereafter the slurry was stirred for another 2 h at 25-30° C. Finally, the product was filtered, washed with DM water (180 ml) and dried under reduced pressure (10-20 mmHg) at 50-55° C. to obtain pure Tipiracil.

Yield: 80 g

Chromatographic purity by HPLC: >99.7%

Polymorph—XRPD represented as FIG. 3

### EXAMPLE—3

### PREPARATION OF 5-CHLORO-6-[(2-IMINO-1-PYRROLIDINYL) METHYL]-2,4(1H, 3H)-PYRIMIDINEDIONE HYDROCHLORIDE (1:1) (TIPIRACIL HYDROCHLORIDE)

Tipiracil pure (50 g; 0.21 mol) was suspended in 1-butanol (500 ml) at 25-30° C. Added concentrate hydrochloric acid (30.5 g; 0.31 mol) at 25-30° C. and stirred the reaction mixture at 50-60° C. for 4-5 h. Thereafter filtered the solid, washed with 1-butanol (50 ml) and dried under reduced pressure (10-20 mmHg) at 60-65° C. to obtain Tipiracil hydrochloride.

Yield: 54 g

Chromatographic purity by HPLC: >99.9%

1-butanol content by 1H-NMR <5000 ppm

Polymorph—Crystal III

### EXAMPLE—4

### PREPARATION OF 5-CHLORO-6-[(2-IMINO-1-PYRROLIDINYL) METHYL]-2,4(1H, 3H)-PYRIMIDINEDIONE HYDROCHLORIDE (1:1) (TIPIRACIL HYDROCHLORIDE)

Tipiracil pure (10 g; 0.04 mol) was suspended in 1-butanol (100 ml) at 25-30° C. Added DM water (5 ml) followed by concentrate hydrochloric acid (6.1 g; 0.06 mol) at 25-30° C. The suspension was stirred for a period of 12-14 h at 25-30° C. Thereafter the solid was filtered, washed with 1-butanol (10 ml) and dried under reduced pressure (10-20 mmHg) at 60-65° C. to obtain Tipiracil hydrochloride

Yield: 11 g

Chromatographic purity by HPLC: >99.9%

1-butanol content by 1H-NMR <1000 ppm

Polymorph—Crystal III

### EXAMPLE—5

### PREPARATION OF CRYSTALLINE 5-CHLORO-6-[(2-IMINO-1-PYRROLIDINYL) METHYL]-2,4(1H, 3H)-PYRIMIDINEDIONE HYDROCHLORIDE (1:1) (TIPIRACIL HYDROCHLORIDE)

5-Chloro-6-chloromethyluracil (10.0 g, 0.05 mol) was suspended in DMF (100 ml) at 25-30° C. under nitrogen atmosphere. Thereafter 2-iminopyrrolidine (12.28 g; 0.15 mol) and sodium ethoxide (14.9 g; 0.15 mol) was added at 25-30° C. The suspension was stirred for a period of 14 hrs at 25-30° C. then filtered the reaction mass at 25-30° C. and dried under reduced pressure. The filtered solid was suspended in DM water (60 ml) and neutralized with acetic acid to (pH—7.0) and stirred for 1 hr at 25-30° C. The solid product was off and washed with DM water (10 ml). The solid was dissolved in 1NHCl (102 ml) and treated with activated charcoal at 25-30° C. for 15 min, then filtered thorough hyflo. The filtrate concentrated under vacuum at 50-55° C. to obtain solid, which was washed with ethanol (30 ml) and dried under reduced pressure to obtain crystalline Tipiracil hydrochloride.

Yield: 3.9 g

Chromatographic purity by HPLC: 99.29

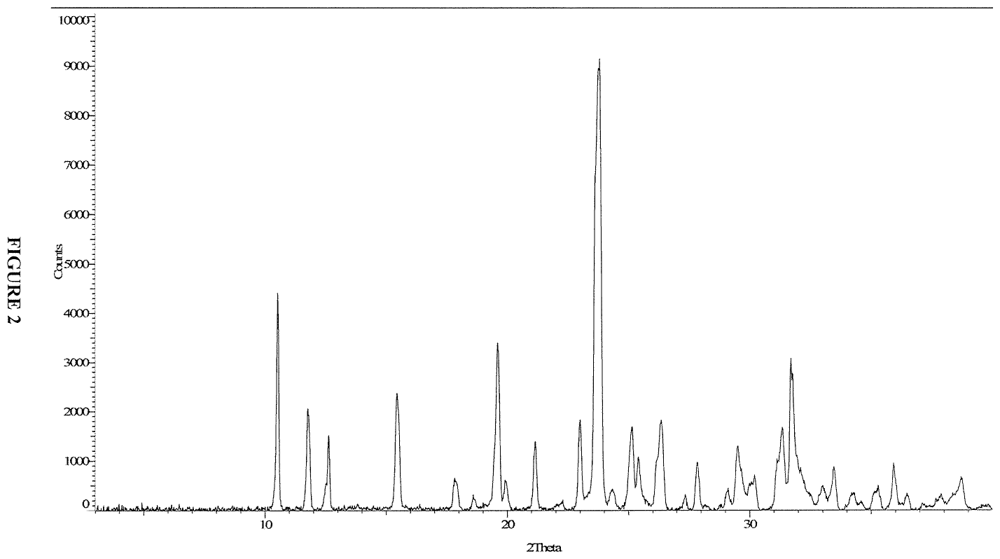

Polymorph—XRPD represented as FIG. 2.

We claim:

1. A process for the preparation of crystal III of Tipiracil hydrochloride substantially free from residual solvent, which comprises reaction of Tipiracil with hydrochloric acid in presence of solvent system comprises alcohol and water.

2. The process of claim 1, wherein the alcohol comprises C₁ to C₆ alcohols.

3. The process of claim 2, wherein the C₁ to C₆ alcohols comprises methanol, ethanol, isopropanol, propanol, 1-butanol, tertiary butanol or mixtures thereof.

4. A process for the preparation of crystal III of Tipiracil hydrochloride substantially free from residual solvent, which comprises reaction of Tipiracil with hydrochloric acid in presence of C₄-alcohol.

5. The process of claim 4, wherein the C₄-alcohol comprises n-butanol, isobutanol or tertiary butanol.

6. A process for the preparation of Tipiracil base having purity greater than about 99.0% by HPLC, which comprises treatment of crude Tipiracil base with an acid followed by a base.

7. The process as claimed in claim 6, wherein the acid comprises organic acid or inorganic acid.

8. The process as claimed in claim 6, wherein the acid is acetic acid.

9. The process of claim 6, wherein the base comprises inorganic base or organic base.

10. The process of claim 6, wherein the base is sodium hydroxide.
